(19) United States
(12) Patent Application Publication
(10) Pub. No.: US 2008/0293048 A1
(43) Pub. Date: Nov. 27, 2008

(54) METHODS AND KITS BASED ON UGT1A7 PROMOTER POLYMORPHISM

(76) Inventors: Christian P. Strassburg, Hannover (DE); Timo O. Lankisch, Hannover (DE); Michael P. Manns, Isernhagen (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/681,984

(22) Filed: Mar. 5, 2007

Related U.S. Application Data
(63) Continuation-in-part of application No. PCT/EP05/09508, filed on Sep. 5, 2005.
(60) Provisional application No. 60/607,297, filed on Sep. 7, 2004.

(30) Foreign Application Priority Data
Sep. 6, 2004 (EP) EP 04021103.9

(51) Int. Cl. C12Q 1/68

(52) U.S. Cl. 435/6

(57) ABSTRACT

The present invention relates to methods for predicting the efficacy, safety and toxicity of substances, e.g. of drugs and prodrugs. Furthermore, the present invention relates to a method for the stratification of mammalians for the treatment of a disease. Moreover, the present invention provides for kits and its use for determining the efficacy, safety and toxicity of substances, in particular of drugs and prodrugs. The present invention allows for the selection of therapeutic regimens utilizing host genetic information, including gene sequence variances. The methods for identification of the specific DNA sequence variations according to the present invention include both in vitro and in vivo approaches.

### CROSS-REFERENCE TO RELATED APPLICATIONS

This application is a continuation in part of the international application PCT/EP2005/009508 which was filed on Sep. 5, 2005, and claims a priority to U.S. Provisional Application 60/607,297 filed on Sep. 7, 2004 and European application EP 04021103.9 filed on Sep. 6, 2004, which are incorporated herein entirely by reference.

### TECHNICAL FIELD

The present invention relates to methods for predicting the efficacy, safety and toxicity of substances, e.g. of drugs and prodrugs. Furthermore, the present invention relates to a method for the stratification of mammalians for the treatment of a disease. Moreover, the present invention provides for kits and its use for determining the efficacy, safety and toxicity of substances, in particular of drugs and prodrugs. The present invention allows for the selection of therapeutic regimens utilizing host genetic information, including gene sequence variances. The methods for identification of the specific DNA sequence variations according to the present invention include both in vitro and in vivo approaches.

### BACKGROUND

Many drugs or other treatments are known to have a highly variable safety and efficacy in different individuals. A consequence of such variability is that a given drug or other treatment may be effective in one individual, and ineffective or not well-tolerated in another individual. Thus, administration of such a drug to an individual in whom the drug would be ineffective would result in wasted cost and time during which the patient's condition may significantly worsen. Also, administration of a drug to an individual in whom the drug would not be tolerated could result in a direct worsening of the patient's condition and could even result in the patient's death.

For some drugs, over 90% of the measurable variation in selected pharmacokinetic parameters has been shown to be heritable. For a limited number of drugs, DNA sequence variances have been identified in specific genes that are involved in drug action or metabolism, and these variances have been shown to account for the variable efficacy or safety of the drugs in different individuals. As the sequence analysis of the human genome is completed, and as additional human gene sequence variances are identified, the power of genetic methods for predicting drug response will further increase.

Medical management of human diseases often present unique medical challenges to clinicians, patients, and caregivers. Many diseases progress and the clinical diagnosis may include more than one disorder, dysfunction, or condition. Furthermore, the efficacy of available treatments may be limited and there may be serious, mostly unpredictable, side effects associated with some drugs. The progressive nature of many diseases makes the passage of time a crucial issue in the treatment process. Specifically, selection of optimal treatment for optimal therapeutic management may be complicated by the fact that it often takes weeks or months to determine if a given therapy is producing a measurable benefit. Thus the current empirical approach to prescribing pharmacotherapy, in which each course of treatment for a given patient is a small experiment, is unsatisfactory from both a medical and economic perspective. Even when an effective treatment is ultimately identified, it often follows a period of ineffective or suboptimal treatment. A method that would help caregivers predict which patients will exhibit beneficial therapeutic responses to a specific medication would provide both medical and economic benefits. As healthcare becomes increasingly costly, the ability to rationally allocate healthcare expenditures, and in particular pharmacy resources, also becomes increasingly important.

Adverse responses to drugs constitute a major medical problem, as shown in two recent meta-analyses (Lazarou, J. et al. Incidence of adverse drug reactions in hospitalized patients: a meta-analysis of prospective studies. JAMA 279:1200-1205, 1998; Bonn, Adverse drug reactions remain a major cause of death. Lancet 351:1183, 1998). An estimated 2.2 million hospitalized patients in the United Stated had serious adverse drug reactions in 1994, with an estimated 106,000 deaths (Lazarou et al.). To the extent that some of these adverse events are due to genetically encoded biochemical diversity among patients in pathways that effect drug action, the identification of variances that are predictive of such effects will allow for more effective and safer drug use.

The UDP-glucuronosyltransferase family of enzymes is a central metabolic system for the glucuronidation of hydrophobic endobiotic and xenobiotic compounds. Glucuronidation leads to the formation of water soluble metabolites which in the majority of cases results in an inactivation of the substrate and the subsequent elimination via bile or urine. The spectrum of possible candidates for this pathway is broad and encompasses steroid hormones, bilirubin and bile acids as well as a vast array of therapeutic drugs, environmental organic substances including known human mutagens. Among the most relevant drugs which undergo glucuronidation are morphine, acetaminophen, chloramphenicol, transplant immunosuppressants such as cyclosporine A and tacrolimus, but also the widely used anti-tumor drug irinotecan active metabolite SN-38. Alterations of glucuronidation activities in the individual are a mechanism by which interindividual profiles of drug metabolism are believed to impact drug efficacy, drug side effects and the predisposition towards environmental mutagen-associated diseases such as cancer.

The human UGT1A proteins have been implicated as risk factors for both the development of cancer and unwanted drug side effects. This risk is determined by 3 differing features of the UGT1A gene locus.

First, the UGT1A gene locus (Genbank accession number AF297093) is regulated and expressed in a tissue specific fashion encompassing the hepatic isoforms UGT1A1, UGT1A3, UGT1A4, UGT1A6 and UGT1A9. In extrahepatic tissues such as mouth, esophagus, intestine, pancreas and colon non-hepatic enzymes (UGT1A7, UGT1A8 and UGT1A10) have been detected conferring a tissue specific profile of glucuronidation to each organ which has been characterized by the analysis of tissue microsomes.

Second, the analysis of different tissues in the human gastrointestinal tract has shown that UGT1A and UGT2B genes are regulated in a polymorphic interindividual fashion leading to differing steady state levels of UGT mRNA, protein and enzymatic glucuronidation activity. The molecular basis of this feature is presently not completely understood.

Third, an increasing number of single nucleotide polymorphisms (SNP) have been identified for all known UGT1A isoforms. For example, WO 99/57322 discloses various polymorphisms of the different UGT1A isoforms. However, data of the genetic analysis and the analysis of the polymorphism are disclosed therein only. No information is provided with respect to the consequences of the various polymorphisms. Furthermore, no information is given for an association of a specific polymorphism with a disease or disorder or enzymatic activity. Today various information have been published relating to a specific polymorphism of an UGT1A isoform which may lead to catalytically altered UGT1A protein variants which will be discussed in more detail below.

Together, this opens the possibility for a considerable number of combinations which represent the biochemical basis of highly interindividual profiles of glucuronidation conserved during evolution. These SNPs mostly lie within the coding regions of the UGT1A gene domains and only few SNPs within the promoter region have been identified to date. However, only few SNPs whether in the coding region, non-coding region or the promoter region are crucial for the difference in drug side effects and the predisposition for various diseases including cancer. In addition, various polymorphisms have been identified influencing the activity of the corresponding isoforms. Further, it has been noted in the past that the presence of a polymorphism in the UGT1A gene does not automatically lead to reduced activity of the enzyme or reduced expression of the enzyme. But only a very limited number of polymorphisms can be associated with specific diseases or physiological reactions.

For example, UGT1A1*28 is characterized by the insertion of a TA into the A(TA)₆TAA element leading to A(TA)₇TAA and a reduction of promoter activity to 30%. This SNP is the genetic basis of Gilbert-Meulengracht's disease leading to unconjugated non-hemolytic hyperbilirubinemia because UGT1A1 is the only efficient metabolic pathway for the elimination of bilirubin from the human body. However, apart from forming the genetic basis of this uncomplicated hepatic disease UGT1A1*28 carrier status has been linked to the susceptibility towards breast cancer and the risk of unwanted intestinal side effects as well as myelotoxicity in colorectal cancer patients treated with irinotecan, a camptothecin analog. The active metabolite of irinotecan, 7-ethyl-10-hydroxycamptothecin (SN-38), which has a 100-1000 fold higher activity than irinotecan, undergoes glucuronidation by UGT1A1 and other UGT. However, the UGT1A1*28 polymorphism is not capable of explaining all cases of irinotecan-associated toxicity.

In addition, polymorphisms in the promoter region of the UGT1A1 enzyme have been described. The outcome of said polymorphisms is varying. That is, while for one polymorphism a reduction of activity is described, the other polymorphism leads to an increase of the activity of the enzyme. Thus, the consequences of polymorphisms within the gene coding for the various UGT1A isoforms are not predictable.

The same is true for polymorphisms occurring in the coding regions of the UGT1A isoforms. For example, in Huang et al., 2002, Pharmacogenetics, 12, 287-297, the identification and functional characterisation of polymorphisms in the UGT1A8 enzyme are described. The polymorphisms analysed in this document were already disclosed in WO 99/57322. Huang et al. demonstrated that out of the three polymorphisms analysed only one resulted in a reduced activity of the enzyme while the two other polymorphisms that no influence on the activity. This is another example, that the consequences of a given polymorphism are not predictable. Thus, a polymorphism occurring in the gene coding for an UGTA1 isoform may lead to an increase or a reduction of the enzyme activity or may have no influence on the activity in a cell or the amount of enzyme present in a cell.

Further, Gagné et al, Mol. Pharmacol, 62, 608-617, 2002 describe the role of various polymorphisms including the above mentioned UGT1A1*28 polymorphism in the metabolism of irinotecan. In particular, it is noted therein that cancer patients presenting UGT1A genotypes as described therein, either alone or in combination to the UGT1A1*28 polymorphism could present significant impaired SN-38 glucuronidating capacity. It is speculated that said patients may present altered response to irinotecan therapy and be at increase risk for adverse reactions. It is particularly emphasized that the isoform UGT1A9 and its polymorphisms may affect the risk for adverse side reactions.

Moreover, it is noted therein that although a high frequency of the UGT1A7 variant alleles in the population and their negative impact on SN-38 glucuronidation is known, their potential association with severe toxicity induced by irinotecan is unlikely. Rather, UGT1A1 and UGT1A9 may represent the crucial isoforms responsible for an increased risk of side reactions during drug therapy.

As mentioned above, irinotecan represents a molecule which is metabolized by UDP-glycoronosyltransferases. Known adverse side effects of irinotecan treatment as anti-tumor drug comprise nausea, diarrhea, vomiting, leukopenia and thrombozytopenia. In particular patients undergoing irinotecan chemotherapy have to stop the therapeutic regimen immediately, thus, worsen individual's condition and protracting the chance of recovery. Therefore, there is still the need for methods allowing stratification of patients undergoing drug therapy before starting the therapeutical regimen in order to determine the most favourable regimen for each patient undergoing drug therapy individually.

Thus, the present invention aims to identify a marker which allow for stratifying most of the individuals undergoing drug therapy. A further object of the present invention is to provide methods predicting the safety, toxicity and efficacy of a substance, in particular of a drug or prodrug in drug therapy, or of a substance released to the environment, i.e. of environmental or occupational poisons. In particular, the object of the present invention is to solve the shortcomings of using the UGT1A1 promoter polymorphism to predict the possibility of adverse side effects in therapeutic regimens and to provide means allowing the identification of almost all individuals having an increased risk of adverse events during drug therapy, e.g. in case of chemotherapy, before starting the regimen.

### DETAILED DESCRIPTION OF THE PRESENT INVENTION

The present invention is generally concerned with the field of identifying an appropriate treatment regimen for a disease based upon a specific genotype of the individual to be treated. It is further concerned with the genetic basis of inter-patient variation in response to therapy, including drug therapy. Specifically, this invention describes the identification of a gene sequence polymorphism useful in the field of therapeutics for optimizing efficacy and safety of drug therapy. This polymorphism or variance may be useful during the drug development process and in guiding the optimal use of already approved compounds. In particular, the DNA sequence variance at position −57 of the promoter region of the nucleotide sequence according to Seq. ID. 1 encoding UGT1A7 is tested in clinical trials, leading to the establishment of diagnostic tests useful for improving the development of new pharmaceutical products and/or the more effective use of existing pharmaceutical products. Thus, the invention relates to methods for identifying individuals, e.g. patient population subsets, which respond to drug therapy with either therapeutic benefit or side effects (i.e., symptomatology prompting concern about safety or other unwanted signs or symptoms).

The inventors have determined that the identification of gene sequence variances at position −57 of the promoter sequence of the UGT1A7 gene which product being involved in drug action are useful for determining drug efficacy and safety and for determining whether a given drug or other therapy may be safe and effective in an individual patient. Provided herein are a sequence variance or polymorphism which is useful in connection with predicting differences in response to treatment and selection of appropriate treatment of a disease or a condition. This polymorphism is useful, for example, in pharmacogenetic association studies and diagnostic tests to improve the use of certain drugs or other therapies.

Moreover, the inventors realized that the combination of the −57 polymorphism and the UGT1A1*28 polymorphism allows the identification of almost all individuals having an increased risk for side reactions, specifically during treatment with irinotecan.

Furthermore, the inventors found that the polymorphism UGT1A1 208 is in linkage disequilibrium with the −57 polymorphism as described herein. Consequently, the 208 polymorphism of UGT1A7 may substitute the −57 polymorphism or may complement the −57 polymorphism.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests which may include, among others, laboratory tests. Symptoms are subjective evidence of disease or patients condition, i.e., the patients perception of an abnormal condition that differs from normal function, sensation, or appearance, which may include, without limitations, physical disabilities, morbidity, pain, and other changes from the normal condition experienced by an individual.

Methods of the present invention which relate to treatments of individuals, e.g. patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “therapy” refers to a process that is intended to produce a beneficial change in the condition of an individual like a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such therapy usually encompasses the administration of a drug, among others.

The term “drug” or “prodrug” as used herein refers to a chemical entity or biological product, or combination of chemical entities or biological products, administered to an individual to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The chemical entity or the biological product may be a xenobiotic or an endogenous substance, preferably, the substance is a drug or prodrug.

The term “drug” may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”.

The term “prodrug” as used herein refers to a precursor of a drug, which is converted into the effective form, i.e. into the drug.

A “low molecular weight compound” has a molecular weight <5,000 Da, more preferably <2500 Da, still more preferably <1000 Da, and most preferably <700 Da.

Thus, in a first aspect, the invention provides a method for the stratification of an individual selecting a treatment for said individual suffering from a disease or condition by determining whether or not a gene in cells of the individual (in some cases including both normal and disease cells, such as cancer cells) contain at least one sequence variance which is indicative of the effectiveness of the treatment of the disease or condition. According to a preferred embodiment, a second sequence variance is identified which is in linkage disequilibrium with the sequence variance according to the present invention, i.e. at position −57 of the promoter sequence shown in Seq. ID.1 encoding UGT1A7.

In some cases, the selection of a method of treatment, i.e. the therapeutic regimen, may incorporate selection of one or more from a plurality of medical therapies. Thus, the selection may be the selection of a method or methods which is/are more effective or less effective than certain other therapeutic regimens (with either having varying safety parameters). Likewise or in combination with the preceding selection, the selection may be the selection of a method or methods, which is safer than certain other methods of treatment in the patient.

The selection may involve either positive selection or negative selection or both, i.e. the selection can involve a decision that a particular method would be an appropriate method to use and/or a decision that a particular method would be an inappropriate method to use. Thus, in certain embodiments the single nucleotide polymorphism at position −57 of Seq. ID. 1 encoding UGT1A7 is indicative that the treatment will be effective or otherwise beneficial (or more likely to be beneficial) in the patient. Stating that the treatment will be effective means that the probability of beneficial therapeutic effect is greater than in a person not having the particular variance(s). In other embodiments, the presence of the sequence variance, i.e. of the single nucleotide polymorphism according to the present invention is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient with a particular variance or variances than in a patient who has a different variance or variances.

The method of selecting a treatment includes excluding or eliminating a treatment, where the presence or absence of the at least one variance is indicative that the treatment will be ineffective or contra-indicated. The phrase “eliminating a treatment” or “excluding a treatment” refers to removing a possible treatment from consideration, e.g., for use with a particular patient based on the presence or absence of the particular variance according to the present invention in cells of that patient, or to stopping the administration of a treatment.

In preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

In particular, according to the present invention the presence of a nucleotide other than T, preferably G, at position −57 of the sequence Seq. ID. 1 encoding UGT1A7 is indicative that the metabolism of a drug or prodrug may be decreased resulting in undesirable side effects, e.g. due to unwanted intoxication of the individual. The decreased metabolism of the drug or prodrug may be based on a reduced activity of the UGT1A7 enzyme in the individual. Alternatively, the decreased metabolism is due to a reduced expression level of enzyme in the cell due to reduced transcription and/or translation. Thus, this may necessitate decreasing the dosage level or the frequency of administration or, alternatively, selecting a different therapy regimen.

Also in this context, a “suitable dosage level” refers to a dosage level that provides a therapeutically reasonable balance between pharmacological effectiveness and deleterious effects. Often this dosage level is related to the peak or average serum levels resulting from administration of a drug at the particular dosage level.

Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects.

The identification of the presence of the particular variance, i.e. the identification of the single nucleotide polymorphism at position −57 of the nucleotide sequence according to Seq. ID. 1 encoding UGT1A7 can be performed in a variety of ways.

Preferably, genomic DNA is used for the identification of said polymorphism. The genomic DNA can be isolated from a sample of the individual's blood by known methods, e.g. by column chromatography and chemical processing. This genomic DNA, which represents the genotype of the individual to be investigated, forms the basis for the methods of the present invention.

The first possibility to determine the presence of the single nucleotide polymorphism according to the present invention, i.e. the polymorphism at position −57 of the nucleotide sequence shown in Seq. ID. 1 and in preferred embodiments additionally of the nucleotide sequence coding for codon 208 according to Seq. ID. 2 and/or the polymorphism in the promoter of UGT1A1 at position −28 involves the amplification of respective fragments of nucleic acids, preferably of genomic DNA e.g. by polymerase chain reaction.

Primers to be used in the amplification step are forward and reverse primer(s) which one binding upstream and one binding downstream of the relevant region. The amplified fragments are preferably about 100 to 300 and more preferably 100 to 150 base pairs in size. The skilled person is well aware how to select suitable primers for amplifying the region of interest.

The amplified nucleic acid product may be sequenced by known techniques. These techniques include the dideoxy termination methods and the use of mass spectrometric methods. The mass spectrometric methods may also be used to determine the nucleotide present at the site of polymorphism.

Alternatively, the polymorphism(s) is/are detected by hybridization methods allowing discrimination of the polymorphism(s). The skilled artisan knows suitable hybridisation methods.

For example, appropriate techniques are allele specific oligonucleotide (ASO) analysis, allele specific PCR (ASP) analysis, restriction fragment length polymorphism (RFLP) analysis, single strand conformation polymorphism analysis (SSCP), heteroduplex analysis, denaturing gradient gel electrophoresis (DGGE), heteroduplex cleavage (either enzymatic as with T4 Endonuclease 7, or chemical as with osmium tetroxide and hydroxylamine) and temperature gradient gel electrophoresis (TGGE), among others.

A further possibility for the identification is based on a PCR-based strategy through specific primer sequences which bind at their 3′ end to the nucleic acid base which is the matter of polymorphism. At the same time, this method can be modified to discriminate between heterozygous and homozygous individuals.

Of course, the polymorphisms at positions 208 of UGT1A7 and the UGT1A1*28 polymorphism can be determined as described above.

As used herein, the terms “effective” and “efficacy” includes both pharmacological efficacy and physiological safety. Pharmacological efficacy refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the presence of one or more sequence variances or alleles.) “Less effective” means that the treatment results in a therapeutically significant lower level of pharmacological effectiveness and/or a therapeutically greater level of adverse physiological effects.

In another aspect, the invention provides a method for selecting a patient for administration of a method of treatment for a disease or condition, or of selecting a patient for a method of administration of a treatment, by comparing the presence or absence of the polymorphism according to the present invention as identified above in cells of an individual to be treated. The presence or absence of said variance is indicative that the treatment or method of administration will be effective and safe in the individual. Depending on the presence or absence of the at least one variance in the individual's cells, the individual is selected for administration of the treatment. In particular, in case the individual display the polymorphism of having a nucleotide other than T, preferably a G, at position −57 of the sequence according to Seq. ID. 1, independent of being homozygous or heterozygous, then it is necessary to decrease the dosage or to select a different therapeutic regimen for said individual.

The invention further provides a method for determining the predisposition to a physiological reaction of an individual to a chemical entity or a biological product which may be administered to an individual to treat or prevent or control a disease or condition. The term “physiological reaction” as used herein refers to a reaction of in the individual. Said physiological reaction encompasses beneficial reactions and adverse reactions. Adverse reactions may be reactions resulting in side effects or may result in effects other than the intended beneficial effect.

In another aspect, the invention provides a kit containing at least one probe or at least one primer (or other amplification oligonucleotide) or both (e.g., as described above) allowing the identification of the polymorphism according to any one of the methods of the present invention. The kit may also contain a plurality of either or both of such probes and/or primers, e.g., 2, 3, 4, 5, 6, or more of such probes and/or primers. It may also be desirable to provide a kit containing components adapted or useful to allow detection of a plurality of variances indicative of the effectiveness of a treatment or treatment against a plurality of diseases. The kit may also optionally contain other components, preferably other components adapted for identifying the presence of the variance according to the present invention. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), and deoxy nucleotide triphosphates (dNTPs). Preferably a probe includes a detectable label, e.g., a fluorescent label, enzyme label, light scattering label, or other label. Preferably the kit includes a nucleic acid or polypeptide array on a solid phase substrate. The array or test arrangement may, for example, include a plurality of different antibodies, and/or a plurality of different nucleic acid sequences. Sites in the array can allow capture and/or detection of nucleic acid sequences or gene products corresponding to different variances in one or more different genes including the variance(s) according to the present invention. Preferably the array is arranged to provide variance detection for a plurality of variances in one or more genes which correlate with the effectiveness of one or more treatments of one or more diseases.

The kit may also optionally contain instructions for use, which can include a listing of at least the variance(s) according to the present invention, correlating with a particular treatment or treatments for a disease or diseases and/or a statement or listing of the diseases for which a particular variance or variances correlates with a treatment efficacy and/or safety.

That means, the present invention provides a kit comprising the genetic detection reagents necessary for at least detecting a single polynucleotide polymorphism at position −57 of Seq. ID. 1 encoding UGT1A7 and instructions for determining the polymorphism.

In particular, the kit according to the present invention allows for conducting the method according to the present invention.

Further, a test arrangement is provided for identifying a single nucleotide polymorphism at position −57 of the nucleotide sequence according to Seq. ID. 1 encoding UGT1A7 comprising the genetic detection reagents necessary for said identification, wherein the nucleotide sequence or any other binding partner necessary for the specific identification of said polymorphism may be fixed on stationary support.

The test arrangement according to the present invention may be for example a DNA-Array system. Thus, the present invention relates further to DNA arrays or DNA chips allowing the identification of the variance(s) according to the present invention using suitable probes and primers.

In preferred embodiments, the kit or test arrangement additionally allow for the identification of further polymorphism(s), in particular of the polymorphism of the nucleotide sequence coding for codon 208 according to Seq. ID. 2 and/or the polymorphism in the promoter of UGT1A1 at position −28.

The invention also includes the use of such a kit or test arrangement to determine the genotype(s) of one or more individuals with respect to the variance sites in one or more genes identified herein. Such use can include providing a result or report indicating the presence and/or absence of one or more variant forms or a gene or genes which are indicative of the effectiveness of a treatment or treatments.

In particular, the present invention relates to the use of the kit or the test arrangement for the stratification of individuals undergoing drug therapy or being exposed to environmental or occupational poisons.

In another aspect, the present invention provides the use of the kit or the test arrangement for the prediction of safety, toxicity and/or efficacy of a substance, in particular of a drug or prodrug in drug therapy.

The present invention provides a number of advantages. For example, the methods described herein allow for use of a determination an individual's genotype, e.g. of a patient's genotype for the timely administration of the most suitable therapy for that particular individual. The methods of this invention provide a basis for successfully developing and obtaining regulatory approval for a compound even though efficacy or safety of the compound in an unstratified population is not adequate to justify approval.

Further, the present invention allows for improving preclinical and clinical development of therapeutics by prospective selection of individuals to be treated. Thus, the prospective screening of individuals reduces the risk of unwanted side effects leading to an increased likelihood of successfully developing and registering a compound or composition of compounds. Hence, the genetic stratification of individual in advance would allow circumventing difficulties normally occurring during clinical development, such as poor efficacy or toxicity for a subset of the tested cohort.

The advantages of a clinical research and drug development program that include the use of polymorphic genotyping for the stratification of individuals, in particular of patients undergoing a drug therapy, for the appropriate selection of candidate therapeutic intervention includes 1) identification of individuals that may respond earlier to therapy, 2) identification of the primary gene and relevant polymorphic variance that directly affects efficacy, safety, or both, 3) identification of pathophysiologic relevant variance or variances and potential therapies affecting those allelic genotypes, and 4) identification of allelic variances or haplotypes in genes that indirectly affects efficacy, safety or both.

Based upon these advantages, designing and performing a clinical trial, either prospective or retrospective, which includes a genotype stratification arm will incorporate analysis of clinical outcomes and potential genetic variation associated with those outcomes, and hypothesis testing of the statistically relevant correlation of the genotypic stratification and therapeutic benefits. If statistical relevance is detectable, these studies will be incorporated into regulatory filings.

In other words, determining the presence of the polymorphism(s) according to the present invention will allow the design of clinical studies being more appropriate to demonstrate the efficacy and safety of the tested compound finally leading to the approval of the compounds or compositions as pharmaceutical products.

Thus, the present invention provides for a method for the stratification of an individual for the treatment of a disease or a condition comprising the step of
(i) identifying a single nucleotide polymorphism at position −57 of the nucleotide sequence according to Seq. ID. 1 encoding UGT1A7.

In particular, the exchange of T to G, C or A is indicative for a reduced metabolic activity of the UGT1A7 enzyme, thus, the conversion of the prodrug to the drug or the degradation of the drug is impaired in comparison to the wild type. Hence, as a consequence the therapeutic regimen has to be adapted accordingly, e.g. by reducing the dosage of the drug or prodrug.

Diseases or conditions being associated with the polymorphism at position −57 of the nucleotide sequence according to Seq ID. 1 encoding UGT1A7 encompasses diseases related to an impaired detoxification and elimination of chemical compounds and natural products by glucuronidation which have a potential for cytotoxicity or genotoxicity and thereby exert inflammatory, mutagenic or toxic effects. These diseases will include unwanted drug reactions in cancer therapy, antibiotic therapy among a multitude of possibilities.

In addition, the method according to the present invention allows for predicting the potential risk of and/or for the diagnosis of carcinomas in the gastrointestinal (colon cancer, pancreatic cancer, hepatocellular cancer, biliary cancer, gastric cancer, esophageal cancer, oropharyngeal cancer) and respiratory (lung cancer) tract and other sites of the human body in addition to chronic inflammatory diseases which include inflammatory bowel disease on the basis said genetic disposition.

In another aspect, the method according to the present invention showing the result of a nucleotide exchange from T to a different nucleotide, in particular to the nucleotide G is regarded as a positive indicator of a sensitivity for carcinomas, in particular for colon, pancreas, hepatic, gastric and esophageal cancer or an inflammatory bowel disease.

In a preferred embodiment, the methods according to the present inventions further comprise the step of identifying a codon exchange at position 208 of the amino acid sequence according to Seq ID. 2 representing UGT1A7. Preferably, said codon exchange at the amino acid level is W to R. On the nucleotide level, the preferred exchange is from T to C at position 1 of codon 208 encoding a tryptophane in the wild type sequence which is altered to an arginine, depicted in the nucleic acid sequence in Seq. ID. 1.

In another preferred method according to the present invention the UGT1A1*28 promoter polymorphism is also identified.

Thus, beside the determination of the single nucleotide polymorphism at position −57 of Seq. ID. 1, the method preferably comprises the detection of additional polymorphisms at position 1 of codon 208 of the UGT1A7 first exon resulting in a codon exchange from W to R and/or the UGT1A1*28 polymorphism, a TA insertion, into the TATA box of the UGT1A1 gene. Since the −57 polymorphism and the 208 polymorphism in linkage disequilibrium, it may be sufficient to determine the 208 polymorphism of the UGT1A7 in combination with the UGT1A1*28 polymorphism.

A further aspect of the present invention relates to a method for screening the efficacy of a drug or prodrug in drug therapy comprising the steps of
(i) providing a first cell or cell line being homozygous for the nucleotide T at position −57 of the nucleotide sequence according to Seq. ID. 1 encoding UGT1A7;
(ii) providing a second cell or cell line being homozygous or at least heterozygous for the nucleotide G, A or C at position −57 of the nucleotide sequence according to Seq. ID. 1 encoding UGT1A7;
(iii) incubating the drug or prodrug with the first and second cell or cell line; and
(iv) determining the capability to metabolize a drug or prodrug of the first and second cell or cell line at the same time point.

Additionally, the present invention provides a method for screening the toxicity and/or safety of a substance comprising the steps of
(i) providing a first cell or cell line being homozygous for the nucleotide T at position −57 of the nucleotide sequence according to Seq. ID. 1 encoding UGT1A7;
(ii) providing a second cell or cell line being homozygous or at least heterozygous for the nucleotide G, A or C at position −57 of the nucleotide sequence according to Seq. ID. 1 encoding UGT1A7;
(iii) incubating the drug or prodrug with the first and second cell or cell line; and
(iv) determining the capability to metabolize a drug or prodrug of the first and second cell or cell line at the same time point.

The determination of the capability to metabolize a substance, e.g. a drug or prodrug may be effected by determining the amount of metabolized substance of the first and second cell or cell line at the same time point. Another embodiment may comprise determining the amount or ratio of dead or living cell in the first and second cell line at the same time. A further embodiment encompasses the determination of the IC50 value of the substance for the first and second cell or cell line. Alternatively, the amount of added substance remaining in the system after a predetermined time may be determined. The determination of living or dead cells, of remaining substance or of metabolized substance may be carried out by commonly known techniques the skilled person is well aware of. The above described techniques allow examining pharmacological kinetics with substances, in particular with drugs and prodrugs, aimed to estimate the metabolism of the substance to be tested.

The cell or cell line usable in the above mentioned methods may be primary cells isolated from an individual or may be cell lines. In particular the cells or cell lines may be genetically engineered cells or cell lines being transient or permanent transformed or transfected with the nucleotide sequences of interest having the specific nucleic acids as mentioned in the sequence allowing the expression of the UGT1A7 enzyme.

Preferred are also humanised, transgenic or conditional animal models containing a gene having the polymorphism described herein, said models are known to the skilled person in the art. In another embodiment, the polymorphic alleles are expressed in heterologous expression systems, e.g. in bacteria, yeast or other eukaryotic cell systems.

Particularly preferred is a method wherein the second cell line being homozygous for a nucleotide other than T, preferably of the nucleotide G at position −57 of the nucleotide sequence according to Seq. ID. 1 encoding UGT1A7.

Also preferred is the use of cells or cell lines having additionally a polymorphism in codon 208 of the amino acid sequence according to Seq ID. 2 and/or the UGT1A1*28 polymorphism, a TA insertion, into the TATA box of the UGT1A 1 gene.

Accordingly, the polymorphism(s) according to the present invention can be used to investigate the metabolism of potentially mutagenic or carcinogenic substances with the aim of making predictions about their toxicity or carcinogenic potency.

UGT enzymes transfer glucuronic acid on suitable substrates. Suitable substrates comprise substances allowing formation of respective glucuronides through hydroxyl, carboxyl, sulfuryl, carbonyl and amino linkages. In particular, substances for the UGT enzymes encompass simple and complex phenols with may be substituted, anthraquinones, flavones and flavanoids, coumarins, C18 steroids, heterocyclic amines, hydroxylated benzo(a)pyrenes, preferably drugs and prodrugs having one or more of these chemical moieties. A particular example thereof is the pharmaceutical drug irinotecan, a camptothecin analog.

### FIGURE LEGENDS

FIG. 1:

Schematic representation of the UGT1A7 gene upstream sequence. Shown is a scheme indicating the localization of five exon polymorphisms and the intron polymorphism located at −57 bp upstream of the UGT1A7 gene ATG codon. The top left panel shows fluorographs of the wildtype and polymorphic sequences. The top right panel shows the results of allelic discrimination PCR analysis (Taqman) capable of discriminating wildtype and polymorphic TATA box variants.

FIG. 2

Allelic discrimination of UGT1A7 exon 1 polymorphisms. Shown are typical examples of the allelic discrimination of the SNPs at codon 129/131 (A) and at codon 208 (B) of the UGT1A7 gene by Taqman PCR.

FIG. 3

Promoter activity by luciferase reporter gene analysis. Shown is the graphic representation of 6 parallel and independent experiments characterizing the ability of wildtype and −57 T>G promoter sequence to drive luciferase expression in transiently transfected HEK293 cells. Luciferase expression is reduced to 30% in the UGT1A7 −57 T>G promoter sequence construct. Results are given as means, error bars indicate standard deviations, all results are normalized for renilla activity and are based on experiments with empty vector as controls.

FIG. 4

UGT1A7 is the principle SN-38 UGT. Autoradiography of a catalytic UGT activity assay using recombinant UGT proteins transiently expressed in HEK293 cells and the irinotecan metabolite SN-38 as substrate. Specific UGT activity is strongest for UGT1A7 which is 5-fold higher than the other activities. Protein amounts were normalized by Western blot (not shown). SN38 GLN, glucuronide of the irinotecan metabolite SN-38.

FIG. 5

FIG. 5 is a scheme showing the various possibilities of known UGT1A7 polymorphisms in the coding region of the UGT1A7 gene.

### EXAMPLES

The following examples will outline the present invention in more detail. However, it is clear that the present invention is not limited by the illustrative examples.

### Patients:

Gilbert-Meulengracht disease: Blood samples were collected from patients diagnosed for the presence of Gilbert-Meulengracht's disease at the Department of Gastroenterology, Hepatology and Endocrinology of Hannover Medical School. In 200 patients (age: 0.4 to 71.3 years, average 17.2 years, 120 male, 80 female) with suspected Gilbert's disease genotyping of the UGT1A1*28 promoter polymorphisms was performed using PCR, direct sequencing and temperature gradient electrophoresis as previously described (Strassburg C P, Vogel A, Kneip S, Tukey R H, Manns M P. GUT, 2002 50; 851-856).

Healthy blood donors: Blood samples were obtained from 427 healthy blood donors from the Department of Transfusion medicine/Blood Bank of Hannover Medical School.

Cancer patients: Five patients with histologically confirmed solid gastrointestinal tumors received irinotecan (Novartis, Switzerland) 80 mg/m² body surface area (30-minute i.v. infusion) in combination with 2 g/m² 5-fluorouracil and 500 mg/m² folic acid once every 3 weeks. Three patients had experienced severe side effects of their therapy; two were without obvious side effects.

Informed consent was obtained from all patients and the study was approved by the Ethics Committee of Hannover Medical School.

### Example 1 Characterization of the UGT1A7 5′ Untranslated Sequence

### Isolation of Genomic DNA

Genomic DNA was isolated from full blood samples by the NucleoSpin Blood XL Kit according to the recommendations of the manufacturer (Machery & Nagel, Dueren, Germany). Concentrations of genomic DNAs were determined by spectrophotometry at 260 and 280 nm. All samples were stored in 10 mM Tris/EDTA buffer (pH 8.0) at −20° C. until analysis.

### PCR Analysis

The UGT1A7 promoter sequence was amplified by PCR. The forward primer (5′-GTACACGCCTTCTTTTGAGGGCAG-3′, Seq. ID. 3) was located from base pair (bp) −103 to −80 downstream of the ATG start codon (see Seq. ID. 1), whereas the reverse primer (5′-TGCACTTCGCAATGGTGCCGTCCA-3′, Seq ID. 4) was located from bp −292 to −315 upstream of the ATG start codon. Sequencing of both primer regions demonstrated no underlying polymorphisms. The 371-bp product was amplified in a volume of 50 μl consisting out of 20 ng genomic DNA, 20 μmol/l of primers, 0.5 μl (5 U/ml) of Biotherm DNA Polymerase (Genecraft, Muenster, Germany) with 5 μl supplied buffer and 0.2 mmol/l of each deoxynucleoside triphosphate. After a hot start at 94° C. for 5 minutes, 32 cycles of 94° C. for 30 seconds, 63° C. for 30 sec followed by 7 min elongation at 72° C. were run on a Perkin Elmer GeneAmp PCR 2400 system (Perkin Elmer, Juegesheim, Germany).

### Sequence Analysis

The PCR products were visualized by 2% agarose gel electrophoresis, purified by using the UltraClean purification Kit (Mobio, Solana Beach, Calif.) and a Sequence PCR was performed by a Dye Terminator Cycle Sequencing Kit 1.1 (Applied Biosystems, Darmstadt, Germany). The nucleotide sequences were determined on an ABI 310 automated sequencer (Applied Biosystems).

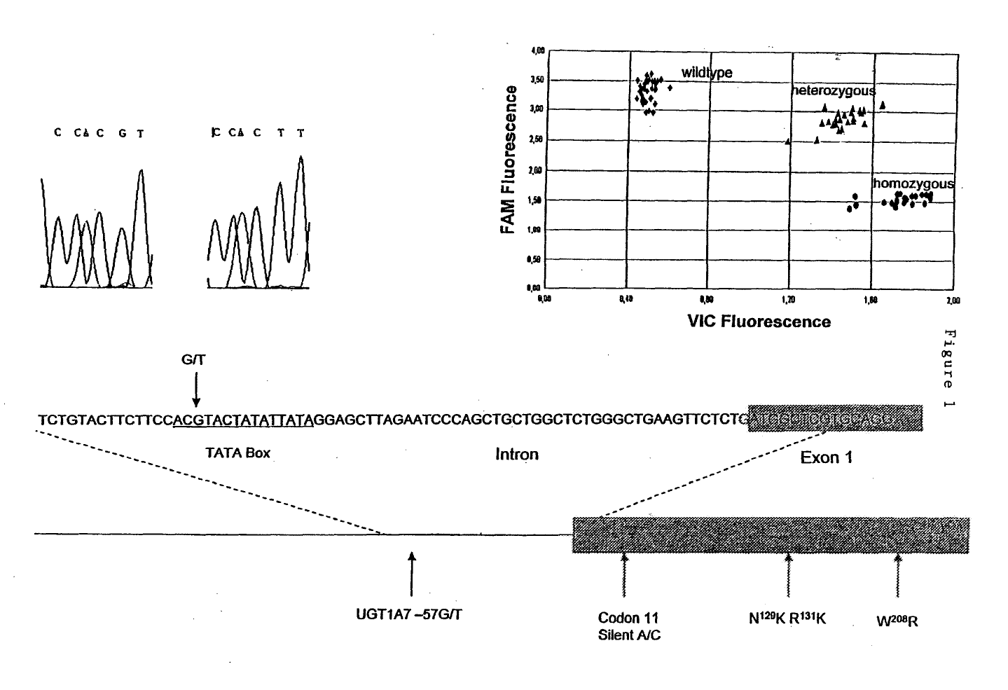

Based upon the genomic DNA sequence deposited in GenBank (accession number AF297093) primers were designed for the amplification of 315 bp upstream of the ATG start codon of the UGT1A7 exon 1 sequence (FIG. 1). The analysis of the obtained sequence suggests that a TATA box for polymerase binding is located between base pairs −59 and −44 from the ATG codon, which is in agreement with the structure of other intron regions at the UGT1A gene locus. The analysis of 427 genomic DNA samples from healthy blood donors identified a single nucleotide transversion from thymidine (T) to guanine (G) at position −57. In contrast to the sequence deposited in GenBank (AF297093), which indicates a G at position −57 our data indicates that the most prevalent variant in our cohort was a T (Table 2 below). The homozygous T (−57 T/T) was detected in 160 (37%) individuals, a heterozygous T (−57 T/G) was present in 203 (48%), and the homozygous G (−57 G/G) was identified in 64 (14%) samples. Based on these findings and in contrast to the GenBank entry AF297093 −57 T appears to represent the wild type sequence with a gene frequency of 0.61 characterized by a single nucleotide polymorphism with a gene frequency of 0.39. Sequence analysis further indicates that this polymorphism affects the TATA box region of the UGT1A7 gene and is only the second TATA box polymorphisms apart from UGT1A1 (UGT1A1*28) identified to date at the human UGT1A gene locus.

### Example 2 Association of the −57 T>G Polymorphism with UGT1A7 Exon 1 Polymorphisms

### Allelic Discrimination Genotyping

Approximately 10 ng of genomic DNA were used as a template in Taqman 5′-nuclease assays for three different SNPs, which were first detected by sequencing. Primers and Probes specific for each SNP were designed with Primer Express software (Applied Biosystems) and labelled with either 6-FAM or VIC as reporter dyes and MGB-NFQ (Applied Biosystems) as quenchers (Table 1). The Taqman assays were performed using 600 nM primer concentrations and 200 nM probe concentrations (Applied Biosystems) and qPCR Mastermix Plus (Eurogentec, Seraing, Belgium). The run consisted of a hot start at 95° C. for 10 minutes and 35 cycles of 94° C. for 15 seconds and 61° C. for 1 min. All assays were performed in 25 μl reactions in 96-well trays using an ABI 7000 instrument (Applied Biosystems).

TABLE 1

Primers for Taqman analysis of UGT1A7 single
nucleotide polymorphisms

| | | |
|---|---|---|
| | UGT1A7 N¹²⁹K R¹³¹K | Seq. ID. No. |
| Forward Primer | 5′-CACCATTGCGAAGTGCATTT-3′ | 5 |
| Reverse Primer | 5′-AGG ATC GAG AAA CAC TGC ATC A-3′ | 6 |
| Probe wildtype | 6-FAM-TAATGACCGAAAATT-MGB | 7 |
| Probe homozygous | VIC-TTAAGGACAAAAAATTAGT-MGB | 8 |
| | UGT1A7 W²⁰⁸R | |
| Forward Primer | 5′-CCAGACTTCTCTTAGGGTTCTCAGAC-3′ | 9 |
| Reverse Primer | 5′-AGACATTTTTGAAAAAATAGGGGCA-3′ | 10 |
| Probe wildtype | 6-FAM-AGGAGAGAGTATGGAAC-MGB | 11 |
| Probe homozygous | VIC-AGGAGAGAGTAGGGAAC-MGB | 12 |
| | UGT1A7-57 T>G | |
| Forward Primer | 5′-TTTTGAGGGCAGGTTCTATCTGTA-3′ | 13 |
| Reverse Primer | 5′-GCAGCTGGGATTCTAAGCTCCTA-3′ | 14 |
| Probe wildtype | 6-FAM-CTTCTTCCACTTACTATATT-MGB | 15 |
| Probe homozygous | VIC-TCTTCCACGTACTATATTA-MGB | 16 |

Previous analyses have identified 5 base pair exchanges at positions 11, 129, 131, and 208 in the first exon of UGT1A7 leading to functionally altered UGT1A7 protein variants designated UGT1A7*1 (wild type), UGT1A7*2, UGT1A7*3 and UGT1A7*4 (Strassburg C P, Vogel A, Kneip S, Tukey R H, Manns M P. GUT, 2002 50; 851-856).

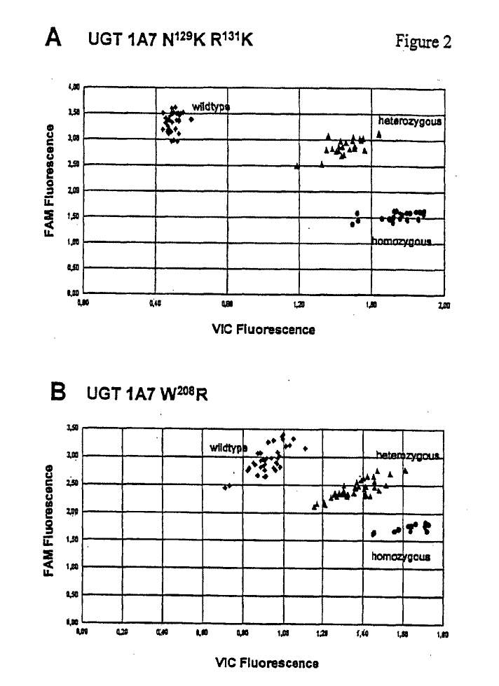

Studies from different laboratories have found that SNPs at 129 and 131 as well as 11 and 208 appear to be in linkage dysequilibrium and always occur in combination. Therefore it was studied whether the intronic polymorphism at −57 bp was associated with the functional SNPs located within exon 1. Taqman allelic discrimination PCR analysis of 427 healthy blood donors was able to precisely discriminate intron and exon SNPs of the UGT1A7 gene (FIGS. 1 and 2). The data show that −57 G was always present when W208R (T to C transition at codon 208) was detected and never found together with exon 1 wildtype sequence. The T to C exchange at codon 208 of the UGT1A7 first exon is present both in the UGT1A7*3 (N129K/R131K and W208R) and UGT1A7*4 (W208R) genotypes (Table 2). The −57 T/G SNP is therefore in linkage dysequilibrium with W208R and thus associated with the UGT1A7*3 and UGT1A7*4 genotypes, which also explains the coincidence of UGT1A7 −57 G with N129K/R131K (Table 2 A). However, in wildtype promoter sequence carriers the association with wildtype N129/R131 is only 40% indicating that only W208R but not N129K/R131K is in linkage dysequilibrium with UGT1A7 −57 G (Table 2, A and B).

TABLE 2
Association of intron and exon polymorphisms
of the UGT1A7 gene
A

| | | N129K/R131K (exon 1) | | |
|---|---|---|---|---|
| UGT1A7 -57 T/G (intron) | | Wildtype | Heterozygous | Homozygous |
| Wildtype | 160 (37%) | 64 (40%) | 75 (47%) | 21 (13%) |
| Heterozygous | 203 (48%) | 0 | 118 (58%) | 85 (42%) |
| Homozygous | 64 (15%) | 0 | 1 (2%) | 63 (98%) |

B

| | | W208R (exon 1) | | |
|---|---|---|---|---|
| UGT1A7 -57 T/G (intron) | | Wildtype | Heterozygous | Homozygous |
| Wildtype | 160 (37%) | 160 (100%) | 0 (0%) | 0 (0%) |
| Heterozygous | 203 (48%) | 0 (0%) | 203 (100%) | 0 (0%) |
| Homozygous | 64 (15%) | 0 | 0 (0%) | 64 (100%) |

Genotyping analyses by Taqman allelic discrimination PCR of the intron SNP, the N129K/R131K (A) and the W208R (B) variants of the UGT1A7 gene first exon indicate a linkage dysequilibrium of the intron located UGT1A7 -57 G SNP and the exon 1 located W208R SNP. W208R was always present when the UGT1A7 -57 G was detected. Conversely, all subjects simultaneously carried both wildtype alleles (W208 and -57 T). A similar linkage dysequilibrium was not found for N129K/R131K although homozygous carriers of UGT1A7 -57G were also homozygous for the UGT1A7 N129K/R131K in all but one individual.

### Example 3 Functionality of the Novel TATA Box Polymorphism of the UGT1A7 Promoter

### Construction of UGT1A7 Luciferase Reporter Gene Vectors

To determine the promoter activity of the 5′ flanking fragment of the human UGT1A7 gene, an approximately 250 bp DNA fragment was amplified by PCR from two different healthy blood donors harbouring either wildtype or polymorphic at −57 downstream of ATG region. The forward primer was designed with a restriction endonuclease site Xho I (5′-ACCGCTCGAGCAGAGAACTTCAGCCCAGAGCC-3′, Seq. ID. 17) and the reverse primer obtained an enzyme restriction site of Kpn 1 (5-′GGAGGTACCAGGGCATGATCTGTCCCCAAGG-3′, Seq. ID. 18). The PCR was performed as mentioned above (PCR analysis) and purified from an 1% agarose gel by a QIA quick gel extraction kit (Qiagen, Hilden, Germany) and then further digested with the restriction endonucleases, Xho I and Kpn 1 (New England BioLabs, Frankfurt, Germany) as recommended by the supplier. The PGL-3 basic vector was digested by Xho I and Kpn 1 as well and dephosphorylated by a shrimp alkaline dephosphatase (Boehringer, Mannheim, Germany). The insert of approximately 250 bp was ligated into the pGL3 basic vector using the fast link ligation Kit (Fermentas, St. Leon-Rot, Germany), followed by transformation into JM-109 cells. Clones were picked and prepared by using the plasmid DNA purification kit (Machery-Nagel). The Sequence of the insert was confirmed by DNA sequencing using the pGL primer 2 rev (5′-CTTTATGTTTTTGGCGTCTTCC-3′, Seq. ID. 19) (Promega, Mannheim, Germany).

### Cell Culture

The human embryonic kidney (HEK) 293 cells were grown on 200 ml culture dishes (Nunc, Roskilde, Denmark) in Dulbecco's modified Eagle's medium (DMEM) supplemented with 10% fetal calf serum (Gibco, Karlsruhe, Germany) and 25 mM glucose. The medium was changed daily and maintained at 37° C. under an atmosphere of 5% CO₂/95% air and cells were harvested after 72 h. The HEK293 cells were seeded into 6-well plates and then incubated for 24 h before transfection. The HEK293 cells were co-transfected with 2 μg of pGL3 −57T or G UGT1A7 promoter vector plasmid and 30 ng PhRL-TK plasmid (Promega, Mannheim, Germany), and incubated at 37° C. under 5% CO₂ culture conditions for 24 hours with Lipofectin (Invitrogen, Karslruhe, Germany) and Optimem (Gibco). The next day 1 ml DMEM was added followed by another 24 h of incubation.

### Luciferase Assays

Cells were harvested after 72 h of induction by washing twice with PBS and then rocked in the presence of 100 μl passive lysis buffer for each well (Promega) for 15 min. All luciferase measurements were made using a Lumat LB 9507 (EG & G Berthold, Bad Wildbad, Germany) according to the manufacturer's instructions (Dual-Reporter Assay, Promega). Firefly luciferase luminescence measurements were normalized to renilla luciferase luminescence measurements before any further analysis. The promoterless pGL3-basic plasmid (Promega, Mannheim, Germany) was used as a control and to normalize the luciferase activities in each separate experiment.

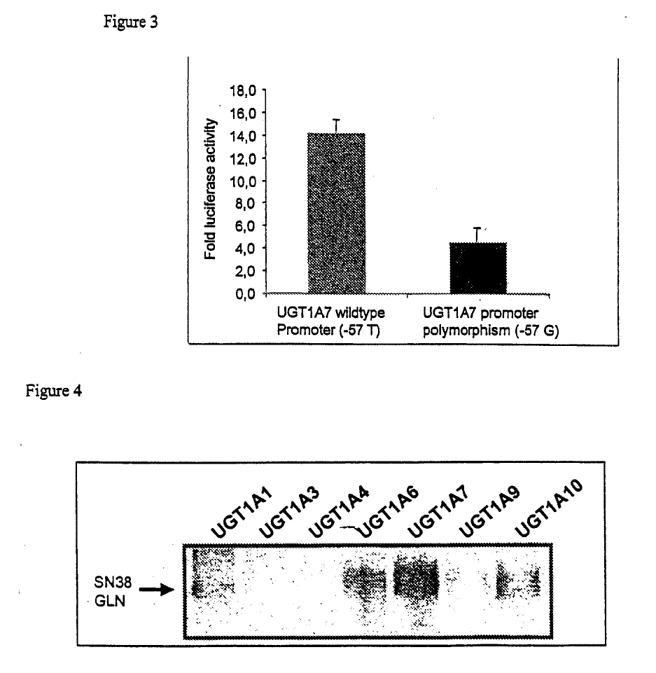

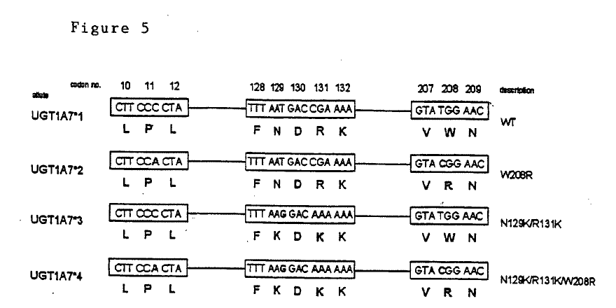

Both wildtype −57 T and variant −57 G promoter sequence carrying 5′ intron sequence fragments of 250 bp were amplified, cloned into the pGL3 firefly luciferase reporter gene plasmid and transfected into HEK293 cells in order to assess their ability to drive luciferase expression and thus determine the functional properties of this single nucleotide change. In six parallel experiments the wildtype UGT1A7 TATA box construct exhibited 14-fold activation of luciferase expression over control (empty plasmid) (FIG. 3). This finding confirms the presence of a promoter element in the −250 bp of the UGT1A7 gene. In contrast −57 G only showed a 4-fold luciferase expression indicating a 70% reduction of promoter activity attributable to the T to G exchange. Transfection efficiencies were controlled and normalized by renilla luciferase activity in each experiment. The identified promoter polymorphism therefore results in a 70% reduction of UGT1A7 promoter activity in comparison to wild type activity.

### Example 4 Irinotecan Metabolite SN-38 is a Substrate of the UGT1A1 and UGT1A7 Proteins

### Catalytic Glucuronidation Assay

UGT1A1, UGT1A3, UGT1A4, UGT1A6, UGT1A7, UGT1A9, UGT1A10 were transiently transfected into HEK293 cells, cells harvested after 72 hours and used as recombinant protein for UGT catalytic activity assays as previously described in detail (Strassburg C P, Nguyen N, Manns M P, Tukey R H. Gastroenterology. 1999; 116:149-60). Protein amounts were normalized by total protein determinations using the Bradford method and were additionally monitored by Western blot using a previously described anti UGT1A antibody directed against the common exon 2 of all UGT1A proteins.

SN-38 represents the active increased activity metabolite of irinotecan and was used as a substrate. The radiography of the activity assay in FIG. 4 demonstrates that UGT1A1, UGT1A6, UGT1A7 and UGT1A10 are identified as relevant isoforms for SN-38 glucuronidation. UGT1A7 showed the highest specific activity with SN-38, which was 5-fold higher than that found with UGT1A1, UGT1A6 and UGT1A10. UGT1A7 therefore represents a relevant isoform metabolizing the anti-cancer drug irinotecan. These data indicates that genetically determined variants of this protein impact irinotecan efficacy and toxicity (FIG. 4).

### Example 5

### Association of the UGT1A7 and the UGT1A1*28 Promoter Polymorphisms

Previous studies have established an association of UGT1A1*28 promoter polymorphism and irinotecan toxicity based upon the catalytic activity of the UGT1A1 protein towards irinotecan and its active metabolite SN-38. In view the findings according to the present invention of a functional promoter polymorphism of the UGT1A7 gene and the in vitro determined highest activity of this UGT1A isoform with SN-38 we analyzed a cohort of 200 patient DNA samples which were genotyped previously for the presence of Gilbert-Meulengracht's disease (Table 3). In this cohort, sequencing and temperature gradient gel electrophoresis using the methods described in the examples above, identified 71 patients homozygous for UGT1A1*28, 65 patients heterozygous for UGT1A1*28, and 64 patients with the wildtype UGT1A1 promoter. Out of the 71 patients who were homozygous for UGT1A1*28 only two displayed a wildtype UGT1A7 promoter (−57 T). The presence of at least one allele of UGT1A7 −57G was therefore 97%. Conversely, individuals with a wild type UGT1A1 promoter had a wildtype UGT1A7 −57T promoter variant in 73%. These data provide evidence for an association of the Gilbert-Meulengracht promoter UGT1A1*28 with the newly identified functional UGT1A7 promoter polymorphism. Both represent the only known examples of functional promoter polymorphisms at the human UGT1A gene locus. The high activity of UGT1A7 toward SN-38 implicates this finding as a risk factor for irinotecan efficacy and toxicity in anti-cancer therapy.

TABLE 3
Association of UGT1A1*28 promoter polymorphisms
with the novel UGT1A7 promoter polymorphism

| UGT1A1*28 | UGT1A7 -57 G/T intron polymorphism | | |
|---|---|---|---|
| (A(TA)₇TAA) | Homozygous | Heterozygous | Wildtype |
| Homozygous (71) | 53 (75%) | 16 (22%) | 2 (3%) |
| Heterozygous (65) | 6 (9%) | 50 (77%) | 9 (14%) |
| Wildtype (64) | 5 (8%) | 12 (19%) | 47 (73%) |

Genotyping of 200 patients referred for suspected Gilbert-Meulengracht's disease. Among these patients 71 were homozygous for the UGT1A7*28 TATA box polymorphism of the UGT1A1 bilirubin transferase gene. Taqman allelic discrimination PCR analysis of all 200 patients demonstrated that in individuals homozygous for the UGT1A1*28 TATA box polymorphism 73% carried the homozygous UGT1A7 −57 G promoter polymorphism, and only 2 (3%) had the wildtype UGT1A7 promoter indicating that among homozygous Gilbert patients 98% carry the reduced activity UGT1A7 promoter polymorphism. However, UGT1A7 −57 T>G is also present in individuals who carry a wildtype UGT1A1 promoter.

### Example 6

### UGT1A7 Promoter Variants in Cancer Patients Treated with Irinotecan Exhibiting Side Effects

Five patients with proven metastatic cancer receiving irinotecan-based chemotherapy were genotyped for the aforementioned UGT1A7 and UGT1A1 gene variants with the methods described in the examples above. Two patients (Table 4, number 1 and 4) without side effects carried both the UGT1A1 and UGT1A7 gene wildtype sequences. Three patients with leukopenia and/or diarrhea (Table 4, number 2, 3, 5) all carried combinations of UGT1A7 intron and exon variants as well as the UGT1A1 promoter variant. In these patients a reduction of irinotecan dose to 75% was necessary. In the case of patient 3 and 5 the 30% activity UGT1A7 −57 G promoter polymorphism coincides with the lowest function UGT1A7*3 allele (N129K/R131K and W208R) and the 30% activity UGT1A1*28. This results in 30% UGT1A1 activity and a prediction of less than 10% UGT1A7 activity. These data illustrate naturally occurring examples of UGT1A promoter and exon SNPs with relevance for drug toxicity predisposition.

TABLE 4
Examples of UGT1A7 and UGT1A1 variants in patients receiving irinotecan.

| | | | UGT1A7 | UGT1A7 | | | |
|---|---|---|---|---|---|---|---|
| Patient | Age | Tumor | N129K/R131K | W208R | UGT1A7 -57T > G | UGT1A1*28 | Side effects |
| 1 I. K. | 68 | Colorectal | Wildtype | wildtype | wildtype | wildtype | none |
| | | carcinoma, | | | | | |
| | | lung and liver | | | | | |
| | | metastases | | | | | |
| 2 J. H. | 69 | gastric carcinoma, | −/+ | −/+ | −/+ | +/+ | Severe |
| | | liver metastases | | | | | leukopenia, |
| | | | | | | | anemia |
| 3 G. H. | 72 | Colorectal | +/+ | +/+ | +/+ | +/+ | Severe |
| | | carcinoma, | | | | | leukopenia, |
| | | lung and liver | | | | | anemia |
| | | metastases | | | | | |
| 4 D. K. | 63 | Colorectal | Wildtype | wildtype | wildtype | wildtype | none |
| | | carcinoma, | | | | | |
| | | lung and liver | | | | | |
| | | metastases | | | | | |
| 5 M. B. | 62 | Colorectal | +/+ | +/+ | +/+ | +/+ | Diarrhea, |
| | | carcinoma, | | | | | anemia |
| | | lung and liver | | | | | |
| | | metastasis | | | | | |

Examples of five patients receiving irinotecan therapy for metastatic gastrointestinal tumors, 2 without side effects and 3 suffering from leukopenia, anemia and/or diarrhea. The combination of UGT1A7 -57 G and the exon SNPs detected in patients 3 and 5 lead to the simultaneous presence of the low activity UGT1A7*3 (N129K/R131K and W208R) allele and the 30% activity UGT1A7 promoter resulting in a predicted UGT1A7 activity of below 10% in addition to a UGT1A1 activity of 30% (UGT1A1*28). +/+, homozygous; −/+, heterozygous.

### Example 7

It is known that the UGT1A7 protein expressed in the extrahepatic gastrointestinal tract exhibits a five-hold higher specific activity with SN-38 than UGT1A1. SNPs at the UGT1A7 gene locus alter enzyme activity and/or transcription. Evidence from the Gunn rat model suggests that intestinal UGTs may play a major role in irinotecan toxicity. However, UGT1A7 gene polymorphisms in the UGT1A7 first exon alone were not found to be associated with irinotecan toxicity in Japanese patients (Ando, M. et al., Jpn. J. Cancer Res, 2002; 93(5); 591-595). In view of these findings the contribution of transcription altering as well as activity altering SNPs of the human UGT1A7 gene were analyzed together with UGT1A*28 in a large cohort of 105 irinotecan-treated patients with metastatic colorectal cancer to identify the potential role of a marker combination with the potential to improve the assessment of the predisposition to irinotecan toxicity.

### Study Subjects:

The analysis reported in the following represents an ancillary assessment of 105 blood samples of patients that had been enrolled in a phase III treatment trial (FIRE; Fluorouracil-Folic acid-Irinotecan-Eloxatin trial) evaluating treatment with irinotecan (CPT-11) plus oxaliplatin versus CPT-11 plus 5-fluorouracil (5-FU) and folic acid (FA). The aim of this study was to assess adverse events, dose reductions and clinical outcome. In both treatment arms 80 mg/m² of CPT-11 were administered intravenously over 30 minutes on day 1, 8, 15, 22, 29 and 36 and the cycle repeated after day 50. In arm A of the FIRE study FA (500 mg/m², 120 min. infusion) and 5-FU (2000 mg/m², 24 h infusion) were co-administered on days 1, 8, 15, 22, 29, 36, and in arm B oxaliplatin (85 mg/m², 120 min infusion) were co-administered on day 1, 15 and 29. At the beginning of each treatment cycle adverse events and clinical symptoms were documented. Patients with known Gilbert's disease were excluded from this study. The following adverse events were analyzed: diarrhea within 24 h and later than 24 after administration of CPT-11, loss of body weight more than 5%, anemia, thrombocytopenia, and leukopenia. The intensity of adverse events was classified according to the WHO-Adverse Reaction Terminology with scores from “0”—“no adverse events” to 4—“life-threatening adverse events”. If the CPT-11 dose had to be reduced to less than 80%, dose reduction was documented at each cycle. Genomic DNA from full blood of all patients was isolated as described before and genotyped after written informed consent was obtained. The study was approved by the local ethics committees.

### Genotyping Analyses:

All genotyping studies were performed in a blinded fashion without prior knowledge of the clinical data of the analyzed patient. Approximately 10 ng of genomic DNA were used as a template in Taqman 5′-nuclease assays. Primers and probes specific for each SNP were designed with Primer Express Software (Applied Biosystems) and labelled with either 6-FAM or VIC as reporter dyes and MGB-NFQ (Applied Biosystems) as a quencher as described in Lankisch et al., Mol. Pharmacol, 2005, 67(5); 1732-1739. The Taqman assays were performed using 600 nM primer concentrations and 200 nM probe concentrations (Applied Biosystems) and qPCR Mastermix Plus (Eurogentec, Seraing, Belgium). The run consisted of a hot start at 95° C. for 10 minutes and 35 cycles of 94° C. for 15 sec. and 61° C. for 1 min. All assays were performed in 25 μl reactions in 96-well trays using an ABI 7000 instrument (Applied Biosystems). UGT1A1*28 was determined by Oncoscreen, Jena, Germany.

### Statistical Analysis:

Patients were classified according to the number of SNPs determined at the UGT1A1*28, UGT1A7 −57 T/G and UGT1A7 N129K/R131K gene loci. In the absence of allelic variants patients were classified as “low risk”, whereas high risk patients were defined if at least one allele of the allelic variant of each analyzed gene locus was present. If only one or two allelic variants were detected at the UGT1A1 or UGT1A7 gene locus, patients were classified as “intermediate risk”. In nine patients the UGT1A1*28 genotyping was not possible for lack of sufficient available DNA. Homozygous UGTA1*28 variants were rare, and heterozygous as well as homozygous UGT1A1*28 variants were summarized in a single group. Frequency and intensity of the adverse events were compared between the risk groups defined above by Cochran-Mantel-Haenszel statistics using modified ridit scores. The risk groups were also compared by multivariant analysis (non-parametric Weil-Lachin procedure) for differences in intensity of 5 adverse events simultaneously. The Mann-Whitney test was used to quantify the difference between the risk groups and the calculate the probability whether a randomly selected patient from one risk group (e.g. “high-risk”) showed more adverse events than a patient from a second risk group (e.g. “low risk”). This test leads to values between 0 and 1 with 0.5 characterizing equal chances, which would indicate no difference in the comparison of these patients. The analysis of incidence and intensity of adverse events was based on the documented 297 treatment cycles involving all 105 patients. The Chi-Square test was used for statistical analysis of CPT-11 dose reductions. P-values were not adjusted for multiple testing. All statistical analyses were calculated by an independent statistician who was not involved in patient treatment or genotyping (Estimate GmbH, Augsburg, Germany).

### Results and Discussion

Gilbert's disease is a well recognized risk factor for the development of irinotecan-associated drug toxicity, in view of the fact that UGT1A1 is involved in SN-38 detoxification. However, reports do not uniformly find a significant association of UGT1A1*28 with irinotecan side effects such as anemia, thrombocytopenia, leukopenia and diarrhea. Therefore, in a first analysis, the presence of UGT1A1*28 alleles in 105 irinotecan-treated patients was studied and associated with adverse events under irinotecan chemotherapy (Table 5 A and B). In this collective neither the development of early (<24 h) or late (>24 h) diarrhea, thrombocytopenia, leukopenia, nor dose reductions below 80% were found to be associated with the presence of UGT1A1*28 alleles. These data derived from one of the largest cohorts of irinotecan-treated individuals to date corroborate data from different studies that have failed to find hematological or gastrointestinal drug toxicity in patients carrying the Gilbert's disease UGT1A1*28 allele, and suggest that additional risk factors may play a permissive role. In an attempt to expand the risk assessment strategy a second analysis was performed aimed at genetic variants of the UGT1A7 gene, which has been demonstrated to exhibit the highest activity with the active irinotecan metabolite SN. In this analysis genotyping included a coding exon 1 variant (N129K/R131K) and a functional promoter variant (−57T/G) of the UGT1A7 gene, both of which have been previously shown to lead to a reduced function UGT1A7 protein or a 70% reduction of UGT1A7 gene transcription, respectively. As observed in the analysis of UGT1A1*28 no significant associations of adverse side affects were found with the UGT1A7 markers alone, again indicating that this genetic trait alone may be a weak predictor of irinotecan-associated toxicity by itself.

Therefore, in a third analysis expanding the hypothesis, the cohort was grouped according to the genotyping data into low, intermediate, and high risk groups based upon the presence of the different UGT SNPs (Table 6) to allow for a genotype-based comparison of different risk groups. This procedure identified 54/105 (51.4%) patients with a high risk genotype based upon the presence of aforementioned UGT variants of the UGT1A1 and UGT1A7 genes. At first sight the high number of variants leading to 51.4% of patients in the high risk group appears unexpected. However, previous reports have documented an association of the UGT1A7*3 genotype, which encompasses N129K/R131K, with colorectal cancer, and the genotyping results found here are in agreement with an association of UGT1A7 SNPs with this disease. When the group combining variants of both UGT1A1 and UGT1A7 (high risk group) was analyzed and compared to those patients with a low risk genotype, the overall incidence of adverse events was significantly higher (p=0.0035, Mann Whitney test: 0.5511, 95% confidence interval: 0.5169-0.5854). Specifically, a significant association of thrombocytopenia and leukopenia with the high risk genotype group was observed (Table 7 A), while the prediction of early and late diarrhea, weight loss and anemia did not reach significance. In the low risk genotype group WHO grade 1 thrombocytopenia (Table 7B) was the highest grade of this adverse event observed. Conversely, grade 2 and 3 thrombocytopenia were observed only in those patients exhibiting the intermediate and high risk genotypes. Similarly, severe leukopenia (Table 7 C) WHO grade 3 and 4 was only observed in the intermediate and high risk groups indicating that leukopenia as well as thrombocytopenia are more prevalent in irinotecan-treated patients with variants of both UGT1A1 and UGT1A7. As would be clinically expected the rate of does reductions to below 80% was significantly higher in individuals characterized by the high risk genotype (high risk group), which demonstrates that the risk associated with a combined genotype involving both UGT1A1 and UGT1A7 was associated with drug toxicity leading to significant treatment consequences in the affected individuals (Table 8). The tumor response was examined in all risks groups, but no association between time to tumor progression or overall survival and risk groups were detected.

From the perspective of drug metabolism the pharmacogenetic association of more than one genetic UGT variant with irinotecan toxicity elucidated in this study is plausible. SN-38 undergoes glucuronidation catalyzed by several UGT1A proteins, specifically by UGT1A1 and UGT1A7. Toxicity is therefore likely to have a higher incidence in individuals exhibiting a UGT haplotype of several functionally relevant SNPs that would act synergistically to reduce SN-38 detoxification. By determining the three markers UGT1A1*28, UGT1A7 N129K/R131K, as well as UGT1A7 −57T/G in this study the group of Gilbert's disease patients (UGT1A1*28) that has been previously recognized as a major risk group for irinotecan toxicity is more precisely defined as evidenced by the lack of association of the individual markers with irinotecan toxicity in this study cohort in contrast to the observed significant association of their combination. These data help to explain the controversial results obtained in other studies analyzing UGT1A1*28 alone and provide a simple tool that improves the prediction of irinotecan-associated drug toxicity. It is interesting to note, that genetic UGT1A7 variants have been found to be associated with colorectal cancer and are therefore more prevalent among colorectal cancer patients. They appear not only to represent a risk factor for the development of cancer but also to increase the risk of drug toxicity during treatment with irinotecan. In the present study colorectal cancer patients with known Gilbert's disease had initially been excluded from the protocol. Our data indicates that among the included patients previously undiagnosed cases of Gilbert's disease were detected, which emphasizes the usefulness of pharmacogenetic testing prior to irinotecan therapy. In view of activities of the drug licensing authorities worldwide aimed at improving drug safety that have recommended pharmacogenetic testing of UGT1A1*28 prior to the initiation of irinotecan therapy, the presented analysis elucidates a refinement of this pharmacogenetic risk by testing for UGT1A1 as well as UGT1A7 SNPs. These tests are capable of detecting the presence of a UGT variant haplotype significantly associated with severe irinotecan side effects, which appears to be superior to determination of the individual markers.

TABLE 5
Analysis of UGT1A1*28 polymorphisms (Gilbert's disease) in irinotecan-treated patients shows
no association drug toxicity including diarrhea, anemia, thrombocytopenia or leucopenia analyzed
by Cochran-Mantel-Haenszel statistics. N denotes treatment cycles as specified in the materials
and methods section (panel A). A relevance of UGT1A1*28 for the necessity of dose reductions
was also not observed (panel B). Similarly, a significant association of UGT1A7 variants with
irinotecan side effects was also not observed (data not shown).
A

| | diarrhea) <24 h) | | diarrhea (>24 h) | | Anemia | |
|---|---|---|---|---|---|---|
| | | UGT1A1*1/*28, | | UGT1A1*1/*28, | | UGT1A1*1/*28, |
| WHO | UGT1A1*1 | UGT1A1*28 | UGT1A1*1 | UGT1A1*28 | UGT1A1*1 | UGT1A1*28 |
| Grade | N (%) | N (%) | N (%) | N (%) | N (%) | N (%) |
| 0 | 70 (71%) | 133 (76%) | 43 (43%) | 71 (41%) | 21 (21%) | 42 (24%) |
| 1 | 17 (17%) | 27 (15) | 32 (32%) | 49 (28%) | 60 (61%) | 98 (56%) |
| 2 | 10 (10%) | 12 (7%) | 17 (17%) | 31 (18%) | 15 (15%) | 31 (18%) |
| 3 | — | 1 (1%) | 5 (5%) | 19 (11%) | 3 (3%) | 3 (2%) |
| 4 | 2 (2%) | 1 (1%) | 2 (2%) | 4 (2%) | — | — |
| All | 99 (100%) | 174 (100%) | 99 (100%) | 174 (100%) | 99 (100%) | 174 (100%) |

| | | | |
|---|---|---|---|
| P value | 0.26 | 0.33 | 0.80 |

A

| | |
|---|---|
| thrombocytopenia | leukopenia |

| | | UGT1A1*1/*28, | | UGT1A1*1/*28, |
|---|---|---|---|---|
| WHO | UGT1A1*1 | UGT1A1*28 | UGT1A1*1 | UGT1A1*28 |
| Grade | N (%) | N (%) | N (%) | N (%) |
| 0 | 82 (83%) | 141 (81%) | 60 (61%) | 91 (52%) |
| 1 | 16 (16%) | 28 (16%) | 26 (26%) | 55 (32%) |
| 2 | — | 5 (3%) | 12 (12%) | 22 (13%) |
| 3 | 1 (1%) | — | 1 (1%) | 4 (2%) |
| 4 | — | — | — | 2 (1%) |
| All | 99 (100%) | 174 (100%) | 99 (100%) | 174 (100%) |

| | | |
|---|---|---|
| P value | 0.67 | 0.18 |

B

| | UGT1A1*28 | |
|---|---|---|
| | −/− | −/+, +/+ |
| Irinotecan dose reduction | N (%) | N (%) |
| <80% | 12 (12%) | 30 (16%) |
| No reduction | 90 (88%) | 153 (84%) |
| Alle | 102 (100%) | 183 (100%) |

| | |
|---|---|
| P value | 0.29 |

TABLE 6
Genotyping results of the UGT1A1 and UGT1A7 genes, and classification
of 105 irinotecan-treated patients. 16 patients were classified as low
risk patients (15.2%), 35 patients as intermediate risk patients (33.3%),
whereas the high risk group comprised 54 patients (51.4%).

| | | | Risk group | | |
|---|---|---|---|---|---|
| UGT1A SNP status | | | | intermediate | |
| | | UGT1A7- | low risk | risk | high risk |
| UGT1A1*28 | UGT1A7^{N}129^{K}/^{R}131^{K} | 57 T/G | N | N | N |
| unknown | −/− | −/− | 2 | — | — |
| | −/+ | −/+ | — | 2 | — |
| | +/+ | +/− | — | 1 | — |
| | | +/+ | — | 4 | — |
| −/− | −/− | −/− | 14 | — | — |
| | −/+ | −/− | — | 13 | — |
| | | −/+ | — | 3 | — |
| | +/+ | −/− | — | 6 | — |
| | | −/+ | — | 2 | — |
| −/+; +/+ | −/− | −/− | — | 2 | — |
| | −/+ | −/− | — | 2 | — |
| | | −/+ | — | — | 21 |
| | +/+ | −/+ | — | — | 16 |
| | | +/+ | — | — | 17 |

| | | | |
|---|---|---|---|
| All | 16 | 35 | 54 |

−/−, wildtype;
−/+, heterozygous;
+/+; allelic variant;
n, number of patients

TABLE 7
Association of the combination of UGT1A1 and UGT1A7 variants with
the overall risk of severe side effects (panel A, univariante analysis).
Patients with the high risk genotype (compare Table 6) were more
likely to develop thrombocytopenia compared to low risk patients
(panel B). Leukopenia was significantly more frequent in the high
risk genotype group than in the low risk genotype group (panel
C), and grade 3-4 leucopenia was only present in high risk patients,
N, number of treatment cycles.
A

| | comparison between risk groups | | |
|---|---|---|---|
| | | low risk vs | |
| | low risk vs | intermediate/high | high risk vs low/ |
| adverse events | high risk | risk | intermediate risk |
| diarrhea (<24 h) | 0.2182 | 0.2039 | 0.3280 |
| diarrhea (>24 h) | 0.2750 | 0.4034 | 0.6647 |
| body weight loss | 0.3555 | 0.3753 | 0.3762 |
| anemia | 0.8041 | 0.7806 | 0.1817 |
| thrombocytopenia | 0.0114 | 0.0132 | 0.6323 |
| leukopenia | 0.0244 | 0.0955 | 0.3983 |

B

| | Risk group | | | | | |
|---|---|---|---|---|---|---|
| Thrombocyopenia | low risk | | intermediate risk | | high risk | |
| WHO Grade | n | % | n | % | n | % |
| 0 | 45 | 96 | 66 | 76 | 131 | 80 |
| 1 | 2 | 4 | 20 | 23 | 27 | 17 |
| 2 | — | — | — | — | 5 | 3 |
| 3 | — | — | 1 | 1 | — | — |
| 4 | — | — | — | — | — | — |
| All | 47 | 100 | 87 | 100 | 163 | 100 |

C

| | Risk group | | | | | |
|---|---|---|---|---|---|---|
| Leukopenia | low risk | | intermediate risk | | high risk | |
| WHO Grade | n | % | n | % | n | % |
| 0 | 33 | 70 | 52 | 60 | 85 | 52 |
| 1 | 10 | 21 | 24 | 28 | 51 | 31 |
| 2 | 4 | 9 | 10 | 12 | 21 | 13 |
| 3 | — | — | 1 | 1 | 4 | 3 |
| 4 | — | — | — | — | 2 | 1 |
| All | 47 | 100 | 87 | 100 | 163 | 100 |

TABLE 8
The presence of all 2 SNPs at the UGT1A1 and UGT1A7 gene
loci (high risk) as associated with a higher probability
of irinotecan dose reductions to below 80%.

| Irinotecan | Risk group | |
|---|---|---|
| dose | low risk | high risk |

| reduction | n | % | n | % |
|---|---|---|---|---|
| <80% | 6 | 8.2 | 32 | 18 |
| no reduction | 67 | 91.3 | 147 | 82 |
| All | 73 | 100 | 179 | 100 |

| | |
|---|---|
| P value | 0.041 |

In other words, the UGT polymorphisms were compared to the occurrence of adverse side events after each cycle of chemotherapeutical treatment. It was found that the presence of the polymorphism at position −57 of UGT1A7 is highly associated with the occurrence of leukopenia. In particular, leukopenia was found with patients being at least heterozygous for the polymorphism −57, i.e. having at least one allele wherein the nucleotide T is substituted with a nucleotide other than T, in particular, being substituted to the nucleotide G.

In addition, the retrospective analysis of the data revealed that in the group of patient having a polymorphism at position −57 of the UGT1A7 gene the physician more decided to reduce the dosage of CPT-11, irinotecan, after a cycle of chemotherapeutical treatment, to an amount of less than 80% of the recommended dosage. In particular, the physician decided to reduce the dosage in individual having at least a heterozygous polymorphism at position −57 of the UGT1A7 gene after 18% of treatment cycles while in the group of patients displaying the wild type sequence at position −57, a reduction of the dosage was effected in 8.2% of the cycles. It is noted that the decision to reduce the dosage rely essentially on the occurrence of adverse side events.

Further, the analysis of the data obtained in this study shows that almost all individuals having an increased risk for adverse reaction can be identified in advance when using a combination of the −57 polymorphism of the UGT1A7 gene and the UGT1A1*28 polymorphism. Thus, the combination of these two polymorphisms enables the physician to stratify the individual for the effective treatment of a disease. Hence, the therapeutic regimen is individualized in advance to avoid or reduce the risk of adverse reaction while receiving the optimum treatment.

Further, since the −57 polymorphism is in a linkage disequilibrium with the 208 polymorphism of the UGT1A7 isoform, the combination of the 208 polymorphism and the UGT1A1*28 will also enable the physician to stratify the individual for a personalized regimen with minimized risk of adverse side effects. Determination of additional polymorphisms, e.g. the 129/131 polymorphism of the UGT1A7 gene or the 115 polymorphism of the UGT1A7 gene strengthens the result obtained by combination of the −57 or 208 polymorphism of the UGT1A7 isoform and the UGT1A7*28 polymorphism.

The above data clearly demonstrates that the polymorphism of the present invention, the −57 polymorphism, in particular in combination with the UGT1A1*28 polymorphism, represents a suitable measure for stratification of patients undergoing drug therapy. That is, the physician can determine the regimen in advance, thus, avoiding the occurrence of adverse side events which may worsen patient's condition.

1. A method for the stratification of an individual for the treatment of a disease comprising the step of
(i) identifying a single nucleotide polymorphism at position −57 of the nucleotide sequence according to Seq. ID. 1 encoding UGT1A7.

2. The method according to claim 1 wherein the presence of a nucleotide polymorphism at position −57 of the nucleotide sequence according to Seq. ID.1 encoding UGT1A7 in at least one allele of the individual is indicative for a regimen for the individual being different to the regimen recommended for the specific pharmaceutical comprising administering lower doses or lower daily dosages of the pharmaceutical.

3. The method according to claim 1 wherein the disease is cancer, neoplasia or chronic inflammatory disease including inflammatory bowel disease, primary sclerosing cholangitis.

4. A method for predicting the efficacy or the toxicity of a drug or prodrug in drug therapy of an individual comprising the step of
(i) identifying a single nucleotide polymorphism at position −57 of the nucleotide sequence according to Seq. ID. 1 encoding UGT1A7 in a DNA sample of said individual,
wherein the exchange of T to a different nucleotide is indicative for a reduction of the metabolizing activity of the enzyme UDP-glucuronosyltransferase.

5. A method of predicting the potential risk of and/or for the diagnosis of carcinomas or chronic inflammatory diseases including inflammatory bowel diseases, primary sclerosing cholangitis on the basis of genetic disposition, characterized in that a DNA sample from an individual to be investigated is tested for the presence of a single nucleotide polymorphism at position −57 of the nucleotide sequence according to Seq. ID. 1 encoding UGT1A7.

6. The method of claim 5 where a positive result of a nucleotide exchange from T to a different nucleotide is regarded as a positive indicator of a sensitivity for carcinomas, in particular for colon, pancreas, hepatic, gastric and esophageal cancer or a chronic inflammatory disease, like chronic inflammatory bowel disease.

7. The method according to claim 1 characterized in that genomic DNA is used for the determination of the polymorphism.

8. The method according to claim 1 wherein the nucleotide polymorphism at position −57 of the UGT1A7 isoform is an exchange of T to G.

9. The method according to claim 1 characterized in that additionally the UGT1A1*28 promoter polymorphism is identified.

10. The method according to claim 1 further comprising the step of identifying a codon exchange at position 208 of the amino acid sequence according to Seq ID. 2 representing UGT1A7.

11. A method for the stratification of an individual for the treatment of a disease comprising at least one step of
i) identifying a single nucleotide polymorphism at position −57 of the nucleotide sequence according to Seq. ID. 1 encoding UGT1A7
ii) determining a polymorphism at position 208.

12. The method according to claim 10 wherein the codon exchange is W to R.

13. A method for screening the efficacy of a drug or prodrug in drug therapy comprising the steps of
(i) providing a first cell or cell line being homozygous for the nucleotide T at position −57 of the nucleotide sequence according to Seq. ID. 1 encoding UGT1A7;
(ii) providing a second cell or cell line being homozygous or at least heterozygous for the nucleotide G, C or A at position −57 of the nucleotide sequence according to Seq. ID. 1 encoding UGT1A7;
(iii) incubating the drug or prodrug with the first and second cell or cell line; and
(iv) determining the capability to metabolize a drug or prodrug of the first and second cell or cell line at the same time point.

14. A method for screening the toxicity and/or safety of a substance comprising the steps of
(i) providing a first cell or cell line being homozygous for the nucleotide T at position −57 of the nucleotide sequence according to Seq. ID. 1 encoding UGT1A7;
(ii) providing a second cell or cell line being homozygous or at least heterozygous for the nucleotide G, C or A at position −57 of the nucleotide sequence according to Seq. ID. 1 encoding UGT1A7;
(iii) incubating the drug or prodrug with the first and second cell or cell line; and
(iv) determining the capability to metabolize a drug or prodrug of the first and second cell or cell line at the same time point

15. A method according to claim 13, wherein step (iv) comprises determining the amount of metabolized substance of the first and second cell or cell line at the same time point.

16. A method according to claim 13, wherein step (iv) comprises determining the amount or ratio of dead or living cell in the first and second cell line at the same time.

17. A method according to claim 13, wherein step (iv) comprises determining of the IC₅₀ value of the substance for the first and second cell or cell line.

18. A kit comprising
the genetic detection reagents necessary for at least detecting a single polynucleotide polymorphism at position −57 of Seq. ID. 1 encoding UGT1A7 and instructions for determining the polymorphism for conducting the method according to claim 1.

19. Test arrangement for identifying a single nucleotide polymorphism at position −57 of the nucleotide sequence according to Seq. ID. 1 encoding UGT1A7 comprising the genetic detection reagents necessary for said identification, wherein the nucleotide sequence or any other binding partner necessary for the specific identification of said polymorphism may be fixed on stationary support.

20. The arrangement according to claim 19 for conducting the method for the stratification of an individual for the treatment of a disease comprising the step of
identifying a single nucleotide polymorphism at position −57 of the nucleotide sequence according to Seq. ID. 1 encoding UGT1A7.

21. Use of the kit comprising
the genetic detection reagents necessary for at least detecting a single polynucleotide polymorphism at position −57 of Seq. ID. 1 encoding UGT1A7 and instructions for determining the polymorphism for conducting the method for the stratification of an individual for the treatment of a disease comprising the step of identifying a single nucleotide polymorphism at position −57 of the nucleotide sequence according to Seq. ID. 1 encoding UGT1A7 or the test arrangement for identifying a single nucleotide polymorphism at position −57 of the nucleotide sequence according to Seq. ID. 1 encoding UGT1A7 comprising the genetic detection reagents necessary for said identification, wherein the nucleotide sequence or any other binding partner necessary for the specific identification of said polymorphism may be fixed on stationary support for the stratification of individuals undergoing drug therapy or being exposed to environmental or occupational poisons.

22. Use of the kit comprising
the genetic detection reagents necessary for at least detecting a single polynucleotide polymorphism at position −57 of Seq. ID. 1 encoding UGT1A7 and instructions for determining the polymorphism for conducting the method for the stratification of an individual for the treatment of a disease comprising the step of identifying a single nucleotide polymorphism at position −57 of the nucleotide sequence according to Seq. ID. 1 encoding UGT1A7 or the test arrangement for identifying a single nucleotide polymorphism at position −57 of the nucleotide sequence according to Seq. ID. 1 encoding UGT1A7 comprising the genetic detection reagents necessary for said identification, wherein the nucleotide sequence or any other binding partner necessary for the specific identification of said polymorphism may be fixed on stationary support for the prediction of safety, toxicity and/or efficacy of a substance, in particular of a drug or prodrug in drug therapy.

23. The method according to claim 4 wherein the nucleotide polymorphism at position −57 of the UGT1A7 isoform is an exchange of T to G.

24. The method according to claim 5 characterized in that additionally the UGT1A1*28 promoter polymorphism is identified.


SEQUENCE LISTING (19 sequences)

SEQ ID NO: 1 - DNA, 2461 bp, homo sapiens
  CDS (129)..(1718)
aatttttttttaaatgaatgaataagtacacgccttcttttgagggcaggttctatctgtacttcttccacgtactatattataggagcttagaatcccagctgctggctctgggctgaagttctctgatggctcgtgcagggtggactggcctccttcccctatatgtgtgtctactgctgacctgtggctttgccaaggcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaatgaccgaaaattagtagaatacttaaaggagagttgttttgatgcagtgtttctcgatccttttgatgcctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattct

SEQ ID NO: 2 - protein, 530 aa, homo sapiens
MARAGWTGLLPLYVCLLLTCGFAKAGKLLVVPMDGSHWFTMQSVVEKLILRGHEVVVVMPEVSWQLGRSLNCTVKTYSTSYTLEDQDREFMVFADARWTAPLRSAFSLLTSSSNGIFDLFFSNCRSLFNDRKLVEYLKESCFDAVFLDPFDACGLIVAKYFSLPSVVFARGIFCHYLEEGAQCPAPLSYVPRLLLGFSDAMTFKERVWNHIMHLEEHLFCPYFFKNVLEIASEILQTPVTAYDLYSHTSIWLLRTDFVLEYPKPVMPNMIFIGGINCHQGKPVPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 3 - DNA, 24 bp, Artificial
  synthetic oligonucleotide primer
gtacacgccttcttttgagggcag

SEQ ID NO: 4 - DNA, 24 bp, Artificial
  synthetic oligonucleotide primer
tgcacttcgcaatggtgccgtcca

SEQ ID NO: 5 - DNA, 20 bp, Artificial
  synthetic oligonucleotide primer
caccattgcgaagtgcattt

SEQ ID NO: 6 - DNA, 22 bp, Artificial
  synthetic oligonucleotide primer
aggatcgagaaacactgcatca

SEQ ID NO: 7 - DNA, 15 bp, Artificial
  synthetic oligonucleotide primer
taatgaccgaaaatt

SEQ ID NO: 8 - DNA, 20 bp, Artificial
  synthetic oligonucleotide primer
tttaaggacaaaaaattagt

SEQ ID NO: 9 - DNA, 26 bp, Artificial
  synthetic oligonucleotide primer
ccagacttctcttagggttctcagac

SEQ ID NO: 10 - DNA, 25 bp, Artificial
  synthetic oligonucleotide primer
agacatttttgaaaaaataggggca

SEQ ID NO: 11 - DNA, 17 bp, Artificial
  synthetic oligonucleotide primer
aggagagagtatggaac

SEQ ID NO: 12 - DNA, 17 bp, Artificial
  synthetic oligonucleotide primer
aggagagagtacggaac

SEQ ID NO: 13 - DNA, 24 bp, Artificial
  synthetic oligonucleotide primer
ttttgagggcaggttctatctgta

SEQ ID NO: 14 - DNA, 23 bp, Artificial
  synthetic oligonucleotide primer
gcagctgggattctaagctccta

SEQ ID NO: 15 - DNA, 20 bp, Artificial
  synthetic oligonucleotide primer
cttcttccacttactatatt

SEQ ID NO: 16 - DNA, 19 bp, Artificial
  synthetic oligonucleotide primer
tcttccacgtactatatta

SEQ ID NO: 17 - DNA, 32 bp, Artificial
  synthetic oligonucleotide primer
accgctcgagcagagaacttcagcccagagcc

SEQ ID NO: 18 - DNA, 31 bp, Artificial
  synthetic oligonucleotide primer
ggaggtaccagggcatgatctgtccccaagg

SEQ ID NO: 19 - DNA, 22 bp, Artificial
  synthetic oligonucleotide primer
ctttatgtttttggcgtcttcc